(19) United States
(12) Patent Application Publication
(10) Pub. No.: US 2016/0345846 A1
(43) Pub. Date: Dec. 1, 2016

(54) Wearable Biomedical Devices Manufactured with Flexible Flat Panel Display Technology

(71) Applicant: Arizona Board of Regents on Behalf of Arizona State University, SCOTTSDALE, AZ (US)

(72) Inventors: Joseph T. Smith, Tempe, AZ (US); Edward J. Bawolek, Chandler, AZ (US); Michael Goryll, Mesa, AZ (US)

(21) Appl. No.: 15/161,481

(22) Filed: May 23, 2016

Related U.S. Application Data
(60) Provisional application No. 62/169,462, filed on Jun. 1, 2015.

(51) Int. Cl. A61B 5/024; H01L 27/32; H05B 33/08

(52) U.S. Cl. CPC A61B 5/02427; H05B 33/0896; H05B 33/0842; H01L 27/3227; A61B 2562/0238; A61B 2562/046; A61B 2562/164; A61B 2562/12

(57) ABSTRACT

A wearable biomedical device manufactured with a flat-panel display technology is provided. The device comprises flexible thin-film layers and a flexible substrate. The layers are laid on the substrate and contain a flexible two-dimensional array of organic light emitting diodes (OLEDs) and photodiodes. This array is connected to an external controller wirelessly or by wire, and the controller controls the pattern of activated OLEDs and photodiodes.

### CROSS-REFERENCE TO RELATED APPLICATIONS

This application claims priority to U.S. Provisional Patent Application No. 62/169,462, filed Jun. 1, 2015, which is incorporated herein by reference as if set forth in its entirety.

### STATEMENT REGARDING FEDERALLY SPONSORED RESEARCH

Not applicable.

### BACKGROUND

Wearable biomedical devices hold the promise of early detection, diagnosis, and treatment of diseases, and can help patients and clinicians by providing non-invasive continuous physiological monitoring in a non-clinical setting before the onset of more severe symptoms. Wearable biomedical devices are typically manufactured using silicon wafer-based microelectronic components bonded to a printed circuit board. However, the devices manufactured using these conventional technologies are rigid and do not conform well with the soft, pliable biological surfaces.

The size of today's flat panel display industry almost defies comprehension. In 2012, flat panel displays were manufactured at a rate of 100 square kilometers per year, which is enough to completely cover one hundred 18-hole golf courses. Even with costs for high-end equipment that can approach several billion dollars, commercial flat panel display factories currently can manufacture displays for less than 10 cents per square centimeter, based on today's HDTV pricing.

Therefore, a pliable biomedical device manufactured with the flat-panel display technology is needed.

### SUMMARY

The present disclosure overcomes the aforementioned drawbacks by providing a wearable biomedical device manufactured with a flat-panel display technology. With this technology, the device is pliable and can be at low cost.

A wearable biomedical device manufactured with a flat-panel display technology is provided. The device comprises flexible thin-film layers and a flexible substrate. The layers are laid on the substrate and contain a flexible two-dimensional array of light emitting diodes (LEDs) or organic light emitting diodes (OLEDs) and photodiodes. This array is connected to an external controller wirelessly or by wire, and the controller controls the pattern of activated OLEDs and photodiodes.

The foregoing and other advantages of the invention will appear from the following description. In the description, reference is made to the accompanying drawings, which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

FIG. 1A is a schematic diagram of an example device configured in accordance with the present application.

FIG. 1B is an exploded view of the schematic cross section of the device placed on the skin of a subject.

FIG. 1C is a schematic of a two-dimensional organic light emitting diode (OLED) array.

FIG. 2A depicts cross section of an OLED array.

FIG. 2B is an exploded view of the layers of cathodes, OLEDs, and anodes.

FIG. 2C shows example curves of the power density of OLEDs versus forward bias voltage.

FIG. 3 is a flowchart illustrating example procedures for manufacturing flexible biomedical devices as disclosed herein.

FIG. 4A shows the placement of an example device, where the photoplethysmograph (PPG) signals are recorded in a transmission mode.

FIG. 4B shows the placement of an example device, where the PPG signals are recorded in a reflection mode.

FIG. 5 depicts an example setup to test the device as disclosed herein.

FIG. 6A depicts example PPG signals acquired in a transmission mode with an example device as disclosed herein.

FIG. 6B depicts example PPG signals acquired in a reflection mode with an example device as disclosed herein.

### DETAILED DESCRIPTION

A wearable biomedical device applying the immense low-cost mass-production capabilities of the flat panel display industry is disclosed. A well-established industrial base already capable of annually supplying an enormous number of consumer electronic products can be tapped to manufacture wearable biomedical devices. As an example, if just 1% of existing flat panel display industrial capacity is diverted to manufacture wearable electronics, approximately 1 billion (˜10 cm²) low cost and ultimately-disposable devices can be manufactured each year. Thus, the flat panel display technology offers a huge opportunity to provide a breakthrough in reducing the cost to manufacture these increasingly-popular devices as well as improve their functionality.

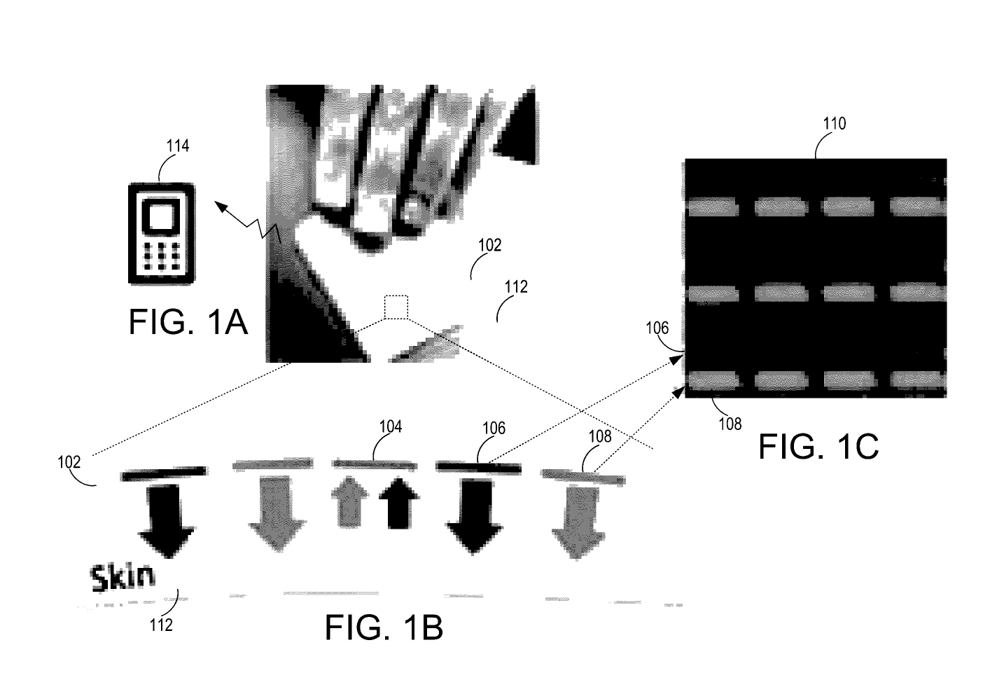

Referring to FIGS. 1A-1C, schematics of an example wearable biomedical device 102 implemented according to the present application are provided. The device 102 can be placed directly on the skin 112 of a subject. Because it is pliable, it conforms closely to the skin of the subject (shown in FIGS. 1A and 1B). The device comprises a two-dimensional (2D) array of organic light emitting diodes (OLEDs) 110 and photodiodes 104. An example photodiode can be a PiN photodiode. Individual OLEDs 106 and 108 can emit different colors of light (e.g., as shown in FIGS. 1B and 1C, 106 is a red OLED and 108 is green). The device 102 communicates with a controller 114 wirelessly or through wire.

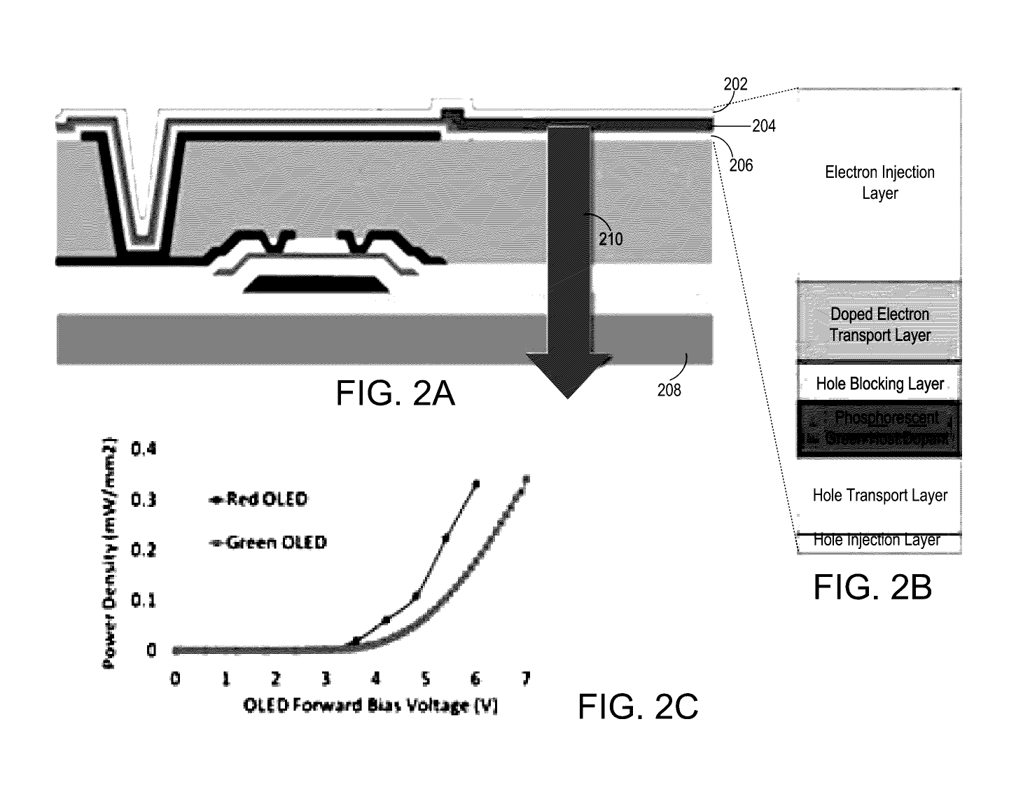

Referring to FIGS. 2A-2C, detailed structure of an example flexible display is provided. FIG. 2A shows the cross section of the flexible display. A flexible display can be thin—approximately the same thickness as a piece of paper—and a typically-transparent sheet of plastic. It can be constructed by sequentially layering and patterning nanoscale thin films. This approach allows the electronics functionality to be built or integrated directly into flexible plastic substrates 208 using thin film components—such as light emitting diodes (LEDs), OLEDs, PiN photodiodes, and thin film transistors (TFTs)—other than separately bonding a large number of discrete electronic components. The example green OLED layer 204 emits green light in the direction towards the bottom of the layer as shown by the green arrow 210 and is sandwiched between a layer of reflective cathodes 202 and a layer of transparent anodes 206. These three layers further comprise thinner layers as shown in FIG. 2B. FIG. 2C shows an example light intensity of red and green OLEDs as a function of forward bias voltage.

To make a display flexible, the same process and tooling currently used to manufacture large commercial flat-panel displays can be used to manufacture the layered structure of the device, except one modification of replacing the starting glass substrate with a flexible plastic substrate temporarily bonded to a rigid alumina carrier. After the thin film process steps are completed, the flexible plastic substrate with the patterned thin film layers on top is then peeled off from the carrier.

Manufacturing with commercial flat panel display technology can also drastically decrease the cost of the devices. Commercial flat panel display technology can manufacture displays on Gen11-sized glass substrates that approach 10 m². This can reduce costs of un-functionalized wearable devices to pennies per cm², which is key for low-cost disposable applications. More importantly, the flat panel display industrial base is well established, can supply massive numbers of large-area electronics components, and thus allow the devices disclosed herein to be rapidly transitioned from laboratory devices to low-cost consumer products.

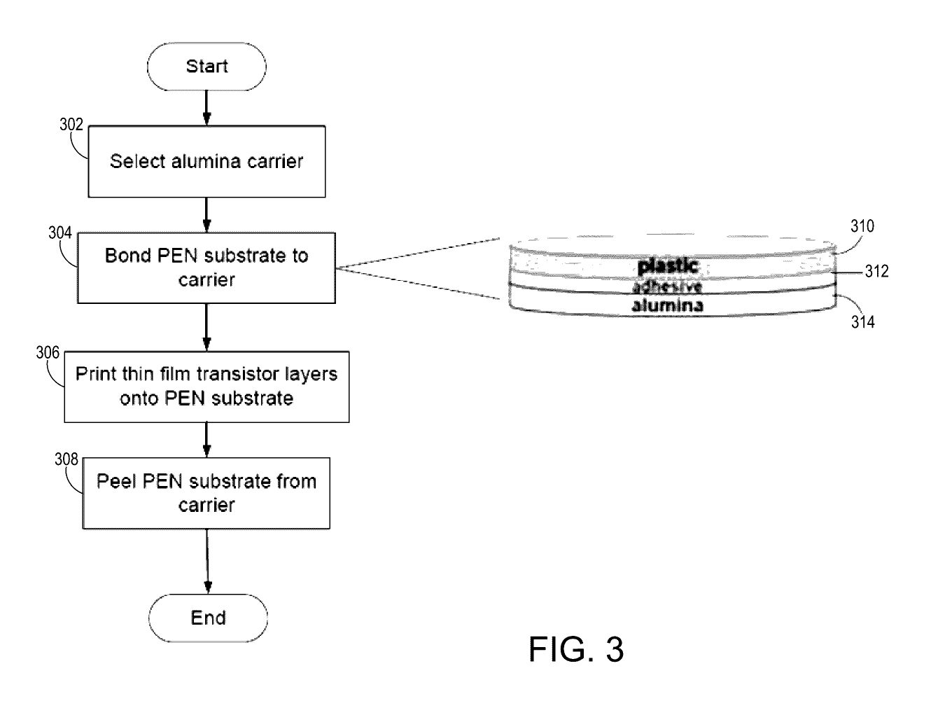

Example processes to manufacture a flexible OLED array are as follows. To make the device flexible, the starting substrate—such as a 125 μm thick DuPont Teijin Films Teonex® polyethylene naphthalate (PEN) flexible plastic substrate—is flexible. Referring to FIG. 3, example processes implemented according to the present application are provided. In step 302, a 6″ rigid alumina carrier is chosen. In step 304, a flexible PEN plastic substrate 310 is bonded with alumina carrier 314 through adhesive 312. In step 306, low temperature (<200° C.) thin film transistor processing is conducted. After the thin film processing is completed, in step 308, the flexible plastic PEN substrate with patterned thin film layers on top is peeled off from the carrier, similar to peeling off a Post-it® note. The rigid alumina carrier allows the flexible display to be manufactured using an off-the-shelf, thin-film semiconductor process tooling without modification.

The flexible photodiodes in the devices as disclosed herein can be manufactured with the manufacturing process for flexible digital x-ray detectors. Additional details on thin film flexible display-based photodiodes and their manufacturing processes can be found in two publications: (i) Smith, J., Marrs, M., Strnad, M., Apte, R., Bert, J., Allee, D., Colaneri, N., Forsythe, E., and Morton, D.: Flexible Digital X-ray Technology for Far-forward Remote Diagnostic and Conformal X-ray Imaging Applications, Proc. SPIE 8730, Flexible Electronics, 2013; and (ii) Marrs, M., Bawolek, E., Smith, J. T., Raupp, G. B., and Morton, D.: Flexible Amorphous Silicon PIN Diode X-ray Detectors, Proc. SPIE 8730, Flexible Electronics, 2013. These two publications are incorporated herein by reference in their entirety.

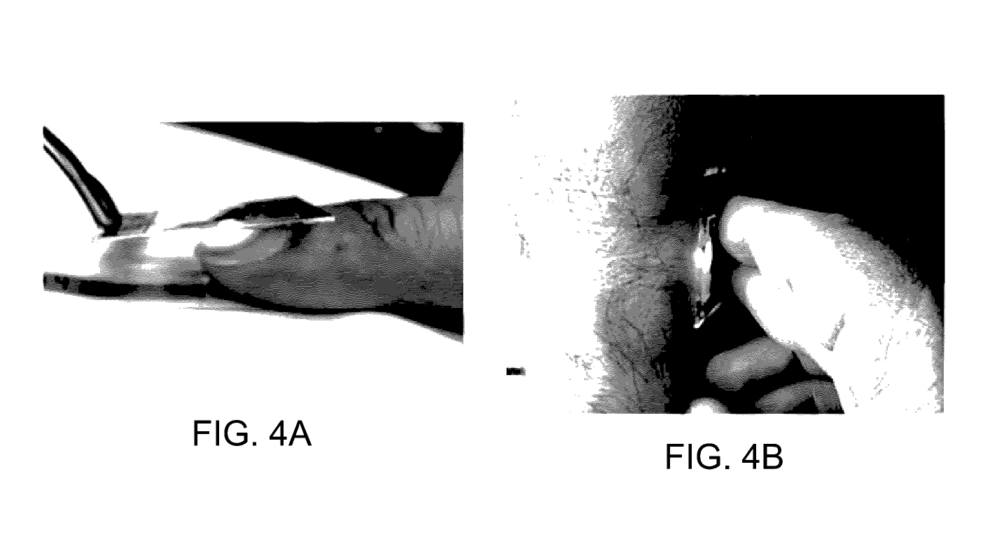

One example of such biomedical devices is a photoplethysmograph (PPG) biosensor using flexible OLED display and thin film photodiode sensor technology for optical heart rate monitoring. PPG detects changes in blood volume in the peripheral vascular system that are directly linked to the activity of the heart. Using this direct relation, an on-skin optical PPG sensor can replace conventional multiple electrode-based ECG methods to accurately measure the heart rate. In addition, the PPG signals are sensitive to vasodilation, which can be detected via particular shapes of the PPG signals. PPG signals can be acquired using an optical transmission probe, similar to a pulse oximeter, as shown in FIG. 4A where example transmission-mode PPG signals are captured by placing an index finger between 620 nm 5 mm² red OLEDs and 6×7 mm a-Si PiN photodiodes. Alternatively, a reflection-based setup can be used to measure PPG, as shown in FIG. 4B where example reflection-mode PPG signals are captured by placing 515 nm green OLEDs and 6×7 mm a-Si PiN photodiodes against surface of the wrist.

The device can be configured in a bandage style as shown in FIG. 1A. Unlike prior attempted flexible PPG biosensors that use experimental printed electronics technology, the devices as disclosed herein are designed for mass production using existing commercial flat panel display tooling.

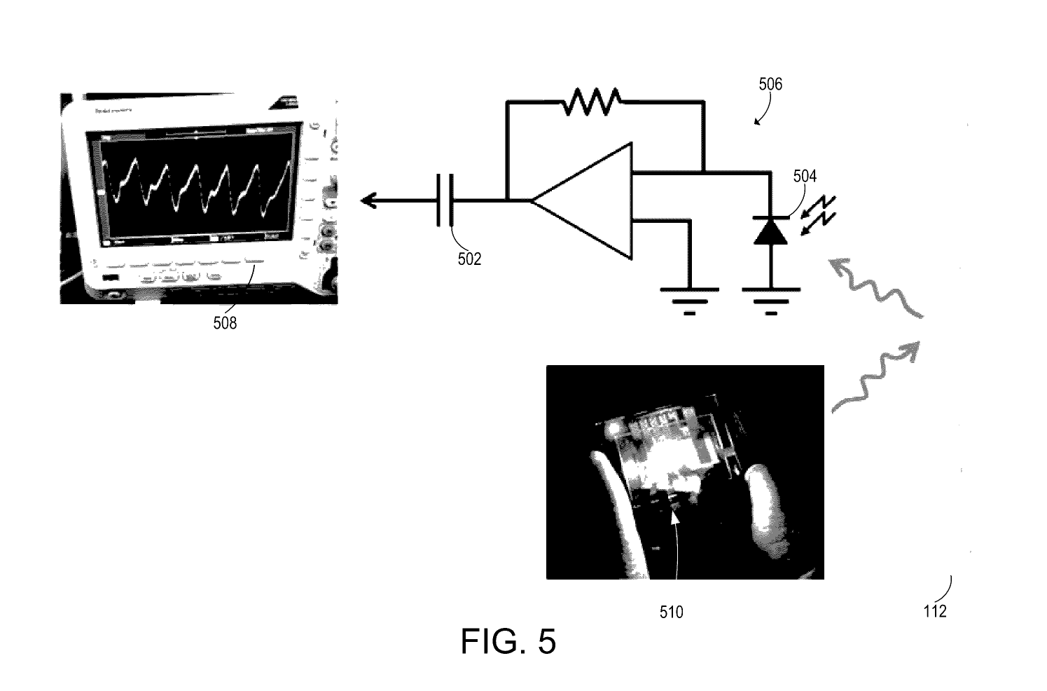

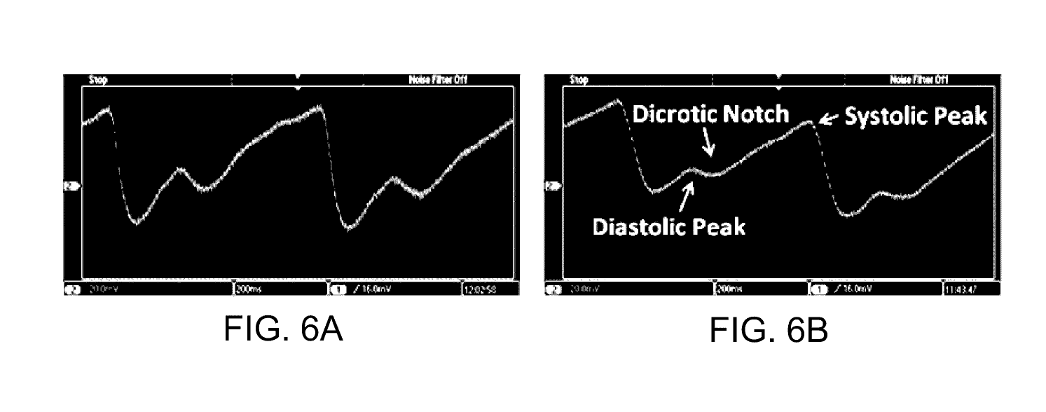

Referring to FIG. 5, a test configuration of an example device is provided. The changes in blood volume can be detected by illuminating the skin 112 with light from flexible OLEDs 510 (e.g., 5 mm² flexible green OLEDs) biased at a certain voltage, e.g., +6 volts DC, and then measuring the amount of AC-modulated light transmitted or reflected to a paired flexible photodiode 104 (e.g., 6×7 mm thin film a-Si flexible PiN photodiodes). The signals can be amplified with a circuit 506 (e.g., LMC6041 optical amplifier configured as a high-gain transimpedance amplifier) and sent to an oscilloscope 508 (e.g., AC-coupled digital oscilloscope). PPG waveforms are then recorded using the oscilloscope. The capacitor 502 blocks the unmodulated background DC signals. As illustrated in FIGS. 6A-6B, recorded transmission-mode (shown in FIG. 6A) and reflection-mode (shown in FIG. 6B) PPG waveforms clearly show the systolic and diastolic peaks, and the dicrotic notch, illustrating that PPG physiological parameters are successfully captured using flexible display and thin film photodiode technology.

However, the intensity of the detected PPG signals can be position dependent, especially in a reflection mode. The maximum signal level is found by positioning the emitter directly over an artery in the wrist where the strongest pulse pressure can be felt. This position dependence limits the widespread adoption of PPG measurements in determining physiological parameters.

To avoid the difficulties associated with having to precisely position a single OLED emitter at the optimal location to detect the strongest PPG signals, a more efficient wearable PPG sensor can instead use a 2D array of OLEDs and photodiodes connected to an external controller 114 as shown in FIG. 1A. Upon startup, an optical pattern search can be activated and the 2D pixel array can cycle through a series of patterns of activated OLED and photodiode pixels while simultaneously monitoring the detected PPG signals to identify and lock in an optimal pattern to maximize the detected PPG signals and simultaneously minimizing the background unmodulated DC signal level by turning off OLED pixels outside the optimal pattern. A 2D checkerboard-style addressed array of alternating OLED and photodiode pixels may significantly improve the functionality of wearable PPG sensors over one-dimensional linear array using discrete components.

The present invention has been described in terms of one or more preferred embodiments, and it should be appreciated that many equivalents, alternatives, variations, and modifications, aside from those expressly stated, are possible and within the scope of the invention. The appended document describes additional features of the present invention and is incorporated herein in its entirety by reference.

We claim:

1. A wearable biomedical device that is manufactured with a flat-panel display technology, comprising:
a) a flexible two-dimensional array of light emitting diodes (LEDs) or organic light emitting diodes (OLEDs) and photodiodes, wherein
the array is connected to an external controller wirelessly or by wire, and
the controller controls a pattern of activated LEDs or OLEDs and activated photodiodes;
b) flexible thin-film layers, wherein the array is contained in the layers; and
c) a flexible substrate, wherein the layers are laid on the substrate.

2. The device as recited in claim 1, wherein the controller controls the pattern of the activated LEDs or OLEDs and the activated photodiodes to maximize detected physiological signals and minimize noise in the physiological signals such that effectiveness of the device is insensitive to a position of the device placed on a subject.

3. The device as recited in claim 2, wherein the device is used to monitor physiological signals of the subject.

4. The device as recited in claim 2, wherein the physiological signals are photoplethysmograph (PPG) signals.

5. The device as recited in claim 4, wherein the PPG signals are recorded in a transmission mode.

6. The device as recited in claim 4, wherein the PPG signals are recorded in a reflection mode.
